(19) United States
(12) Patent Application Publication
(10) Pub. No.: US 2015/0157749 A1
(43) Pub. Date: Jun. 11, 2015

(54) Reusable Grocery Bag Disinfection Machine

(71) Applicants: Roger Taylor Madsen, Palm Desert, CA (US); Winifred Ann Bond-Madsen, Palm Desert, CA (US); William Coleman Bond, Aurora, CO (US)

(72) Inventors: Roger Taylor Madsen, Palm Desert, CA (US); Winifred Ann Bond-Madsen, Palm Desert, CA (US); William Coleman Bond, Aurora, CO (US)

(21) Appl. No.: 14/102,806

(22) Filed: Dec. 11, 2013

(51) Int. Cl. A61L 2/07

(52) U.S. Cl. CPC A61L 2/07

(57) ABSTRACT

The introduction of reusable grocery bags has lead to contamination of these bags by bacteria, virus and E-coli. A steam sterilization and disinfection device is described including a chamber that will contain the reusable grocery bag while it undergoes steam cleaning and disinfection. The reusable grocery bag will be fitted. over a mandrel that will direct the steam to the inside surfaces of the bag. The cleaning chamber will have a cover door (10) such that the bag will be enclosed during the cleaning process. Additional attributes include 2 steam generators (2), electronic control system (5), water tank (3) supplying the steam generators, high pressure pump (7) to charge the steam generators, vacuum pump (9) to evacuate the steam after cleaning and disinfection, and refuse water tank (4) to contain the contaminated water after cleaning.

### RELATED U.S. APPLICATION DATA

U.S. CL. 134/35: 134/37

### REFERENCES CITED

U.S. PATENT DOCUMENTS

| | | | |
|---|---|---|---|
| 4,414,037 | November 1983 | Friedheim | 134/35 |
| 4,807,319 | February 1989 | Poitevin | 15/21 |
| 5,634,501 | June 1997 | Walshe | 141/92 |
| 5,894,014 | April 1999 | Houston et al. | 422/295 |
| 5,915,071 | June 1999 | Harbin, III | 392/401 |
| 6,299,076 | October 2001 | Sloan et al | 239/136 |
| 8,316,505, B2 | November 2012 | Chow | 15/320 |
| 2002/0078977 A1 | June 2002 | Manne | 134/25.2 |
| 2004/0042943 A1 | March 2004 | Wanselin | 422/295 |
| 2010/0306928 A1 | December 2010 | Kimm et al | 8/142 |
| 2012/0222706 A1 | September 2012 | Pears et al | 134/18 |

### BACKGROUND OF THE INVENTION

The present invention relates generally to the disinfection art. It finds particular application in steam sterilization, disinfection and cleaning of reusable grocery bags and will be described with particular detail therein.

More particularly, the present invention provides a medium size disinfection device which contains its own steam generator and is operable on a standard electrical power supply. Provisions are provided for collection of the spent steam within the unit. The disinfection and sterilization device is designed for installation in grocery stores.

Reusable grocery bags can be cleaned and disinfected by laundering them in any standard washing machine. In this regard, most consumers forget to perform this simple task and arrive at the market with a contaminated bag. The apparatus provided isolates the bag in a closed container with high temperature steam environment for a period of time to complete disinfection.

Historically most steam sterilization devices have had special installation requirements. The steam is supplied by industrial boiler and as a such the source of steam was dedicated power source. They did not connect to standard utilities. As more small and remote providers have developed in the industry, the demand for lower cost easily installed steam cleaning devices has dramatically increased.

The present invention provides a novel disinfection device which provides for the safety of the operator and installation flexibility.

For example the steam cleaning devices of the type described in U.S. Pat. Nos. 4,441,4037; 5,634,501; 5,915,071 herein incorporated by reference provide examples of steam cleaners of the type generally related to the present invention.

### SUMMARY OF THE INVENTION

The primary attribute of the present invention is a steam cleaning and disinfection device having a chamber for receiving reusable grocery bags to be decontaminated. The chamber includes a mandrel inside a sealed space, a door which allows loading and unloading of bags from the chamber and insulation surrounding said chamber to protect operators from hot steam and surfaces. The steam cleaning and disinfection device shall be stainless steel construction for easy maintenance by store employees. The control panel shall be mounted near the door and shall provide detailed operating instructions and status of the disinfection process. The door covering the reusable grocery bag disinfection mandrel will be power operated such that it cannot be opened during the disinfection cycle. The mandrel and the its container shall be fabricated from a plastic with a low thermal conductivity value so that it will not retain the heat after the cleaning cycle is complete and will cool down quickly. Thermal sensors shall monitor the disinfection cycle to ensure that complete disinfection has occurred. In addition sensors shall monitor the temperature of the machine so as to avoid exposing the operator to hot surfaces. A vacuum pump is provided to evacuate the spent steam from the cleaning chamber and discard the water in the refuse water tank. The mandrel will contain a sufficient number of small holes such the steam will be distributed uniformly over the entire surface of the reusable grocery bag.

A high pressure pump shall supply distilled water to the steam generators and valves will connect the steam supply from the steam generators to the disinfection mandrel. When the door is open to insert or remove a bag the valve shall be locked out to prevent steam from escaping from the machine. The mandrel shall extend from the machine during loading and unloading to facilitate placement of the bag over the mandrel. Having the mandrel extended will allow the operator to place the bag over the mandrel maintaining a smooth surface and avoiding having folds occur that would prevent full disinfection of the interior surface of the bag. The bag will be held in place by vacuum as the mandrel is pulled into the machine.

Further advantages of the invention will become apparent to those of ordinary skill in the steam cleaning art upon reading and understanding the following detailed description of the invention.

### BRIEF DESCRIPTION OF THE DRAWINGS

The invention may take form in various components and arrangements of components and in various steps and arrangements of steps. The drawings are only for purposes of illustrating a preferred embodiment and are not to be considered as limiting the invention.

FIG. 1 is a front elevation view of the inventive disinfection device with a cover cut open removed;

FIG. 2 is a right side elevation view with panels of the disinfection device cut open and the chamber door in closed position;

FIG. 3 is a left side elevation view with panels of the disinfection device cut open having the closed chamber door;

FIG. 4 is a partial front elevation cut open of the disinfection machine with the door opened and the mandrel extended;

FIG. 5 is a schematic of the disinfection machine components and their connection to each other

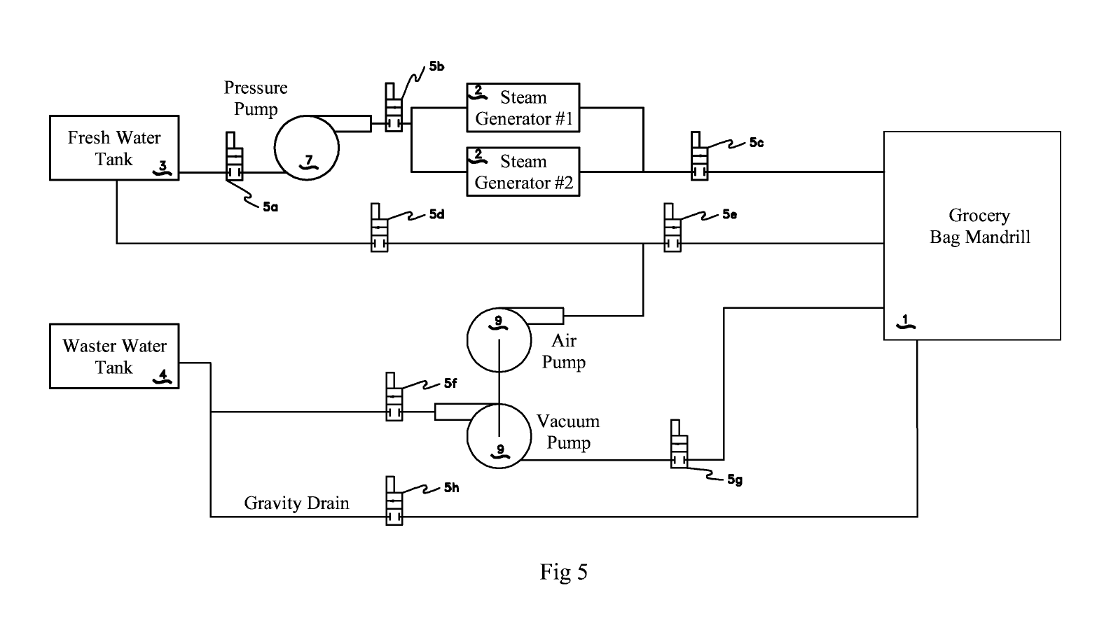

### DETAILED DESCRIPTION OF THE INVENTION

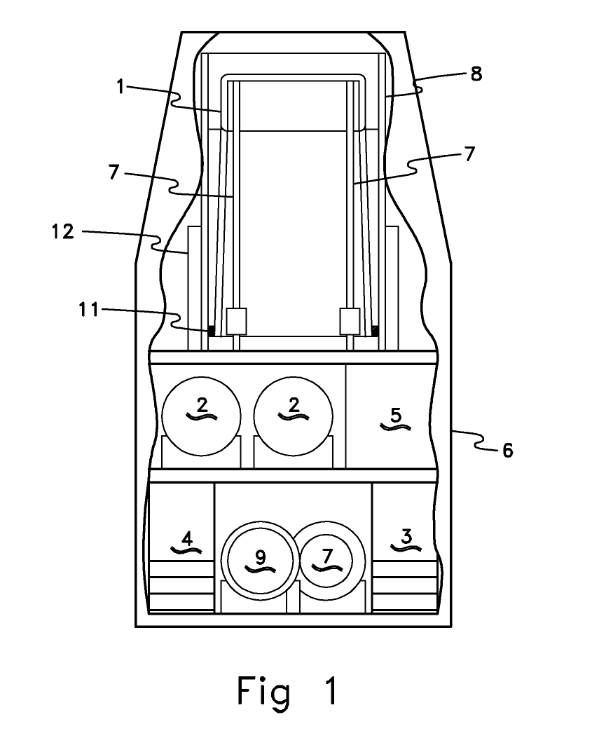

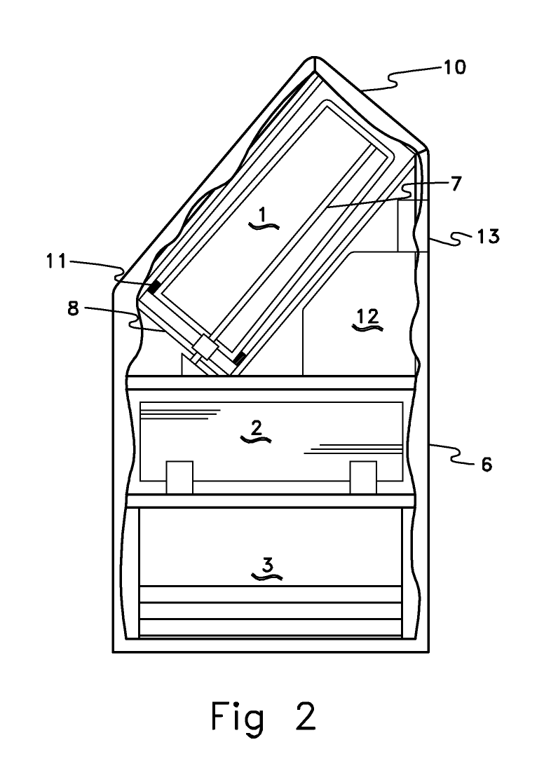

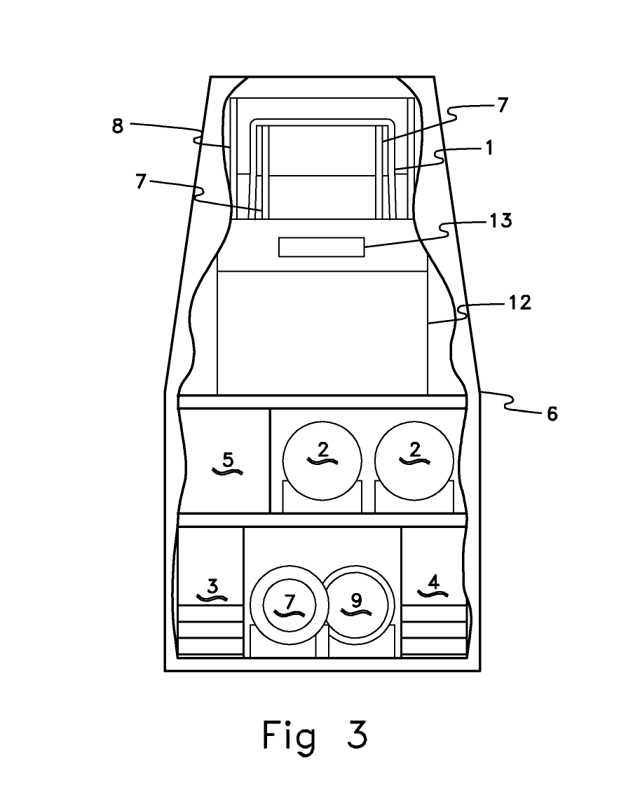

Turning now to the drawings, wherein like elements are identified with like numerals throughout the figures, an embodiment of the invention is shown in FIG. 1, in its configuration as a disinfection machine and in FIGS. 2 and 3 in right and left side views. While the invention will be described in conjunction with a preferred embodiment it will be understood that it is not intended to limit the invention to that embodiment. It is intended to cover all alternatives modifications and equivalents as may be included within the spirit and scope of the invention defined by the appended claims.

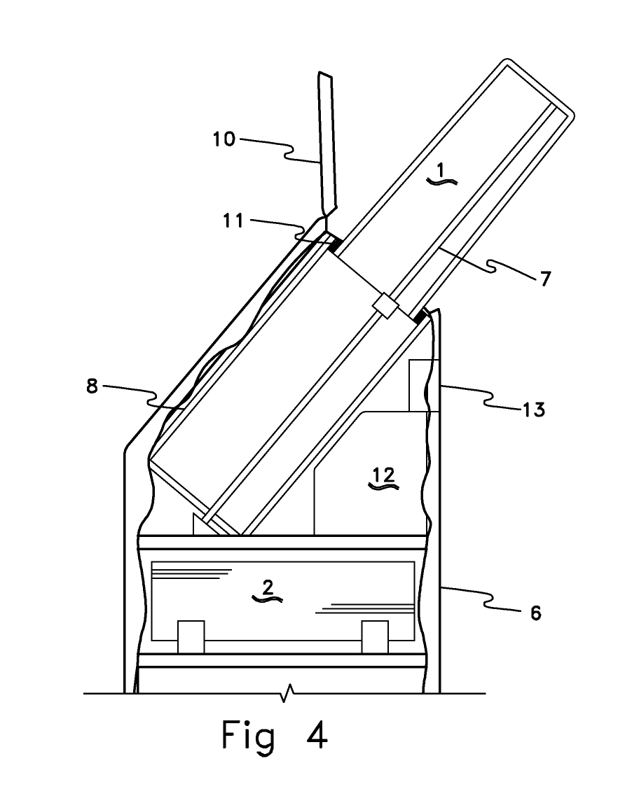

Referring now to FIGS. 1, 2 and 3 in which like numbers are used to represent like elements the inventive disinfection machine (6) is depicted. The disinfection mandrel (1) includes a chamber (8) surrounded by the body of the machine (6). Steam from the supply source (2) enters the chamber (1) through an inlet in the base and exits through myriad of small pin holes designed to distribute the steam uniformly to the interior of the bag being disinfected. The disinfection mandrel (1) is supported by four guides (11) at the bottom of chamber and the extension mechanism (7) that moves the mandrel out to the loading position (FIG. 4) and in to the cleaning position. Items (5a),(5b) and (5c) are the valves required to supply the steam to the mandrel. Valves also located in item (5) can provide vacuum or air pressure to the mandrel from the Air Pump (9) and vacuum pump (9). Fresh water from the tank (3) is pumped by the high pressure pump (7) into the steam generators (2) and the valve (5c) supplies the steam to disinfect the bag when the unit is closed. The vacuum pump (9) is connected by valves in (5) to the chamber (8) to extract the spent steam and or water and dispose of it in the waste water tank (4).

The door (10) is hinged to the main housing (6) of the disinfection machine. The door (10) operating mechanism is linked to the mandrel extending mechanism (7) such that the door will unlatch and will only be opened when the mandrel begins to extend. Travel of the door is controlled by a spring linkage system driven by the extension mechanism so that the door is wide open as the mandrel is extended. Door is designed to open to 90 degrees (FIG. 4). The door has a seal to prevent steam from escaping during the disinfection cycle.

The extending mechanism (7) can be either lead screw design or hydraulic piston. The mechanism shall be capable of a travel of approximately 20 inches. Clear space around the mandrel shall be no less than ½ inch with rounded surfaces so that the bag being disinfected will not be caught on the machine.

Electronic module (12) shall control all functions of the machine and monitor sequence of operation. The control panel (13) provides for initiating the disinfection cycle and for limited user control of the cycle. The primary goal of the machines design is to protect the user from harm or exposure to hot steam.

The following is a brief description of the operation of the machine for one disinfection cycle from start to finish:
a) User approaches machine with a reusable grocery bag to be disinfected, to begin press the “START”.
b) The machine initiates the open cycle, the door is unlatched and swings opens and the mandrel extends to accept the bag.
c) User places their grocery bag over the mandrel pulling it down tight.
d) User then presses the “BEGIN CYCLE”.
e) The machine starts the vacuum pump which pulls the bag tight against the mandrel.

f) The machine retracts the mandrel to inside the machine.
g) The user is then directed to close the door and depress it such that it latches in the closed position.
h) The machine shuts off the vacuum pump.
i) The machine pumps water into the steam generators and steam is forced through the mandrel to the inside of the bag. Disinfection Occurs.
j) Steam is shut off and vacuum pump starts.
k) Air from vacuum pump is forced through mandrel to cool and dry bag. Condensate is collected by vacuum pump and disposed of in the waste water tank.
l) When the temperature of the bag is within safe range the machine unlatches the door and extends the mandrel with a clean bag.
m) User removes their bag.

Therefore it is apparent there has been provided in accordance with the invention a disinfection device that fully satisfies the objects aims and advantages as described above. The invention is described with specific embodiments. It is evident that many alternatives modifications and variations are apparent to those skilled in the art provided by the foregoing description. Accordingly it is intended to cover any and all such alternatives modifications and variations as well within the spirit and broad scope of the intended claims.

We claim:

1. A steam disinfection machine for reusable grocery bags comprised of a chamber including a movable mandrel, mechanical extension mechanism, sealing door, steam generators, pressure pumps, vacuum pumps, air pumps, water tank, waste water tank, and electronic control system, powered by standard utilities, electric and water.

2. The device of claim 1 wherein the user must pay for single use is incorporated.
